(19) United States
(12) Patent Application Publication
(10) Pub. No.: US 2024/0342461 A1
(43) Pub. Date: Oct. 17, 2024

(54) SHEATHS INCLUDING INBUILT MALE LUER CONNECTORS

(71) Applicants: Boston Scientific Medical Device Limited, Galway (IE); Boston Scientific Scimed, Inc., Maple Grove, MN (US)

(72) Inventors: Neeraj Kumar Sharma, Delhi (IN); Qian Liu, Plymouth, MN (US)

(73) Assignees: Boston Scientific Medical Device Limited, Galway (IE); Boston Scientific Scimed, Inc., Maple Grove, MN (US)

(21) Appl. No.: 18/633,001

(22) Filed: Apr. 11, 2024

Related U.S. Application Data
(60) Provisional application No. 63/458,803, filed on Apr. 12, 2023.

(51) Int. Cl. A61M 60/17; A61M 25/00; A61M 39/10; A61M 60/216; A61M 60/419

(52) U.S. Cl. CPC A61M 60/17; A61M 25/0097; A61M 39/10; A61M 60/216; A61M 60/419

(57) ABSTRACT

A sheath for receiving a medical device includes a hub and a body portion. The body portion is configured to be disposed in a patient. An access conduit is coupled to the hub. A stopcock is coupled to the access conduit opposite the hub. The stopcock includes a first leg coupled to the access conduit, a second leg having a male luer connector, and a third leg having a female luer connector.

### CROSS REFERENCE TO RELATED APPLICATIONS

This application claims the benefit of U.S. Provisional Patent Application Ser. No. 63/458,803, filed Apr. 12, 2023, which is incorporated herein by reference.

### TECHNICAL FIELD

The present disclosure relates to sheaths for delivering intravascular medical devices. More specifically, the present disclosure relates to sheaths for constructing bypass blood flow circuits.

### BACKGROUND

In procedures for delivering intravascular medical devices, an introducer sheath is inserted into a blood vessel of a patient, such as the femoral artery, and one or more medical devices are inserted into the introducer sheath and advanced in the blood vessel. In some situations, relatively large medical devices, such as blood pumps, are delivered via introducer sheaths. Such devices are typically delivered by large bore introducer sheaths. However, such sheaths can inhibit blood flow in the vessel and thereby cause ischemia.

### SUMMARY

In an Example 1, a sheath for receiving a medical device includes a hub configured to receive the medical device. A body portion is coupled to the hub and configured to be disposed in a patient. The body portion is configured to receive the medical device from the hub. An access conduit is coupled to the hub. A stopcock is coupled to the access conduit opposite the hub. The stopcock includes a first leg coupled to the access conduit, a second leg having a male luer connector, and a third leg having a female luer connector.

In an Example 2, the sheath of Example 1, wherein the hub includes a port configured to receive the medical device.

In an Example 3, the sheath of Example 2, wherein the port is a first port, and the hub further includes a second port.

In an Example 4, the sheath of any of Examples 1-3, wherein the stopcock further includes a stopcock lumen defined within the first leg, the second leg, and the third leg, and the stopcock further includes a lever being pivotable to selectively isolate one of the first leg, the second leg, and the third leg from the others of the first leg, the second leg, and the third leg.

In an Example 5, the sheath of any of Examples 1-4, wherein the body portion has a size of 16 French.

In an Example 6, the sheath of any of Examples 1-4, wherein the body portion has a size of 6 French.

In an Example 7, the sheath of any of Examples 1-6, wherein the hub includes a hub lumen configured to receive the medical device.

In an Example 8, the sheath of any of Examples 1-7, wherein the body portion includes a body portion lumen configured to receive the medical device.

In an Example 9, the sheath of any of Examples 1-8, wherein the access conduit includes an access lumen.

In an Example 10, a medical device assembly includes a first sheath having a first hub and a first body portion coupled to the first hub. The first body portion is configured to be disposed in a patient. A first access conduit is coupled to the first hub. A first three-way stopcock is coupled to the first access conduit. The first three-way stopcock includes a male luer connector. The medical device assembly further includes a second sheath having a second hub and a second body portion coupled to the second hub. The second body portion is configured to be disposed in the patient. A second access conduit is coupled to the second hub. A second three-way stopcock is coupled to the second access conduit. The second three-way stopcock includes a female luer connector configured to be coupled to the male luer connector of the first sheath.

In an Example 11, the medical device assembly of Example 10, wherein the first body portion has a first size, the second body portion has a second size, and the second size is less than the first size.

In an Example 12, the medical device assembly of Example 11, wherein the first size is 16 French and the second size is 6 French.

In an Example 13, the medical device assembly of any of Examples 10-12, further including a medical device configured to be advanced through the first body portion.

In an Example 14, the medical device assembly of Example 13, wherein the medical device is a blood pump.

In an Example 15, the medical device assembly of any of Examples 10-13, wherein the female luer connector is a first female luer connector, and the second three-way stopcock further includes a second female luer connector.

In an Example 16, a sheath for receiving a medical device includes a hub having a hub lumen and a port coupled to the hub lumen. The port and the hub lumen are configured to receive the medical device. A body portion is coupled to the hub and configured to be disposed in a patient. The body portion includes a body portion lumen coupled to the hub lumen, and the body portion lumen is configured to receive the medical device. An access conduit is coupled to the hub, and the access conduit includes an access lumen coupled to the hub lumen. A stopcock is coupled to the access conduit opposite the hub. The stopcock includes a stopcock lumen coupled to the access lumen, a first leg coupled to the access conduit, a second leg including a male luer connector, and a third leg including a female luer connector.

In an Example 17, the sheath of Example 16, wherein the port is a first port, and the hub further includes a second port.

In an Example 18, the sheath of Example 16, wherein the stopcock lumen is defined within the first leg, the second leg, and the third leg, and the stopcock further includes a lever being pivotable to selectively isolate one of the first leg, the second leg, and the third leg from the others of the first leg, the second leg, and the third leg.

In an Example 19, the sheath of Example 16, wherein the body portion has a size of 16 French.

In an Example 20, the sheath of Example 16, wherein the body portion has a size of 6 French.

In an Example 21, a medical device assembly includes a first sheath having a first hub and a first body portion coupled to the first hub. The first body portion is configured to be disposed in a patient. A first access conduit is coupled to the first hub. A first three-way stopcock is coupled to the first access conduit. The first three-way stopcock includes a male luer connector. The medical device assembly further includes a second sheath having a second hub and a second body portion coupled to the second hub. The second body portion is configured to be disposed in the patient. A second access conduit is coupled to the second hub. A second three-way stopcock is coupled to the second access conduit. The second three-way stopcock includes a female luer connector configured to be coupled to the male luer connector of the first sheath.

In an Example 22, the medical device assembly of Example 21, wherein the first body portion has a first size, the second body portion has a second size, and the second size is less than the first size.

In an Example 23, the medical device assembly of Example 22, wherein the

first size is 16 French and the second size is 6 French.

In an Example 24, the medical device assembly of Example 21, further including a medical device configured to be advanced through the first body portion.

In an Example 25, the medical device assembly of Example 21, wherein the female luer connector is a first female luer connector, and the second three-way stopcock further includes a second female luer connector.

In an Example 26, a sheath assembly includes a first sheath and a second sheath, the first sheath having a first body portion and a first stopcock, the first stopcock including a male luer connector, and the second sheath having a second body portion and a second stopcock, the second stopcock including a female luer connector. A method of using the sheath assembly includes: positioning the first body portion of the first sheath in a blood vessel of a patient; positioning the second body portion of the second sheath in the blood vessel of the patient; coupling the first sheath and the second sheath via the male luer connector of the first sheath and the female luer connector of the second sheath; receiving blood from the blood vessel in the first body portion of the first sheath; providing blood from the first sheath to the second sheath via the first stopcock and the second stopcock; and providing blood from the second body portion of the second sheath to the blood vessel.

In an Example 27, the method of Example 26, further including advancing a medical device through the first body portion of the first sheath.

In an Example 28, the method of Example 27, further including advancing the medical device to a target cardiac location.

In an Example 29, the method of Example 28, wherein the target cardiac location is the left ventricle.

In an Example 30, the method of Example 27, wherein the medical device is a blood pump.

In an Example 31, the method of Example 26, wherein positioning the first body portion of the first sheath in the blood vessel of the patient includes positioning the first body portion in a retrograde manner.

In an Example 32, the method of Example 26, wherein positioning the second body portion of the second sheath in the blood vessel of the patient includes positioning the second body portion in an antegrade manner.

In an Example 33, the method of Example 26, wherein the first body portion has a first size, the second body portion has a second size, and the second size is less than the first size.

In an Example 34, the method of Example 33, wherein the first size is 16 French and the second size is 6 French.

In an Example 35, the method of Example 26, wherein the blood vessel is the femoral artery.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a transverse sectional view of an introducer sheath inserted into a blood vessel, in accordance with embodiments of the present disclosure.

FIG. 2 is a cross sectional view of the introducer sheath of FIG. 1 inserted into the blood vessel and a medical device inserted into the introducer sheath, in accordance with certain embodiments of the present disclosure.

FIG. 3 is a perspective view of the introducer sheath of FIG. 1.

FIG. 4 is a side view of the introducer sheath of FIG. 1.

FIG. 5 is a side view of a stopcock of the introducer sheath of FIG. 1.

FIG. 6 is a side view of a medical device assembly including the introducer sheath of FIG. 1 and antegrade access sheath, in accordance with certain embodiments of the present disclosure.

FIG. 7 is a diagram of an illustrative method of using a sheath assembly to deliver a medical device to a target location in a patient, in accordance with certain embodiments of the subject matter disclosed herein.

While the invention is amenable to various modifications and alternative forms, specific embodiments have been shown by way of example in the drawings and are described in detail herein. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Large bore introducer sheaths can inhibit blood flow in blood vessels and thereby cause ischemia. As a result, physicians typically use a second sheath with antegrade access to construct a bypass circuit. Such a circuit permits blood to flow through the sheaths and return to the blood vessel downstream from the large bore introducer sheath. However, such bypass circuits can be cumbersome to construct and typically require use of a separate connector, such as a male-to-male luer connector, for connecting female luer connectors of the sheaths. Accordingly, certain embodiments of the present disclosure are directed to sheaths, sheath assemblies, and methods that facilitate constructing bypass blood flow circuits via use of both male and female luer connectors.

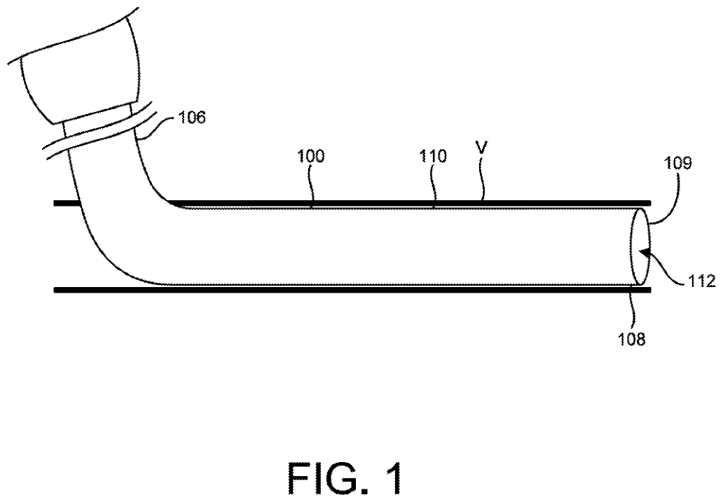

FIG. 1 illustrates a transverse sectional view of a blood vessel V with a sheath 100, such as an introducer sheath 100, partially inserted into the blood vessel V. In some embodiments, the introducer sheath 100 facilitates passage of various relatively large medical devices, such as blood pumps, as will be described further herein, through the introducer sheath 100 and into the blood vessel V. Accordingly, the introducer sheath 100 may be referred to as a large bore introducer sheath. The introducer sheath 100 includes a proximal end portion 106 and a distal end portion 108 opposite the proximal end portion 106. The introducer sheath 100 includes a proximal opening (not shown) adjacent the proximal end portion 106 and a distal opening 109 adjacent the distal end portion 108. A body portion 110 of the introducer sheath 100 extends between the proximal end portion 106 and the distal end portion 108, and the body portion 110 defines a body portion lumen 112. The introducer sheath 100 also includes a hub 120 at the proximal end portion 106. During use, the hub 120 remains outside of the patient, receives one or more medical devices, and facilitates advancing the medical devices through the body portion lumen 112 and the blood vessel V. As described in further detail herein, the hub 120 includes one or more openings or ports for receiving medical devices. The hub 120 also facilitates hemostasis. That is, the hub 120 inhibits blood loss from the body of the patient.

In some embodiments, the sheath 100 may be a repositioning sheath instead of an introducer sheath.

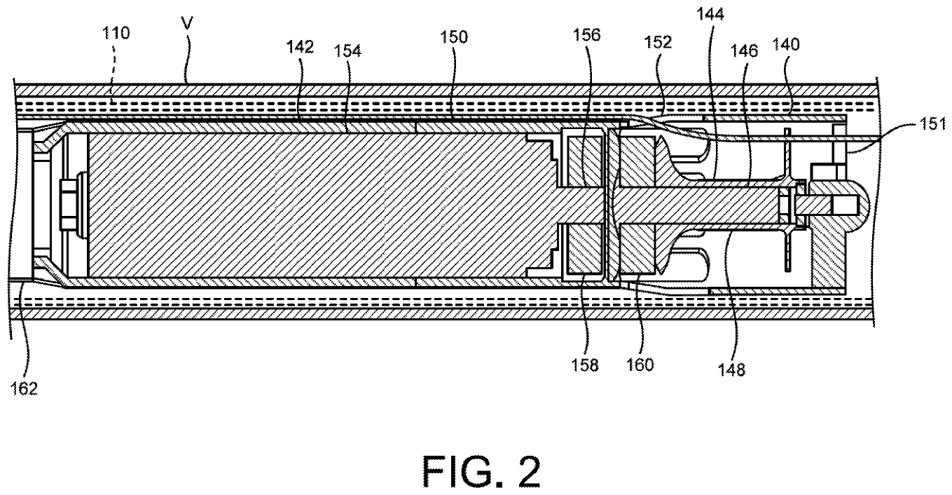

FIG. 2 illustrates a transverse sectional view of the body portion 110 of the introducer sheath 100 upon insertion of a medical device, illustratively a blood pump 150, into the introducer sheath 100. The blood pump 150 may be advanced through the blood vessel V and positioned in a target location, more specifically a target cardiac location such as the left ventricle, via the introducer sheath 100. The blood pump 150 generally includes an impeller assembly housing 140 and a motor housing 142. In some embodiments, the impeller assembly housing 140 and the motor housing 142 may be integrally or monolithically constructed. The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow into a blood inlet 151 of the impeller assembly housing 140, through the impeller assembly housing 140, and out of a blood outlet 152 of the impeller assembly housing 140. In some embodiments the impeller shaft 146 and the impeller 148 may be integrated, and in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula (not shown), and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. Additionally, as illustrated in FIG. 2, a catheter 162 extends from a proximal end of the blood pump 150. In some embodiments, the catheter 162 may be coupled to the motor housing 142 through a tapering connector and/or various other connecting means. The catheter 162 may have a flexible construction to facilitate to the delivery of the blood pump 150. While the introducer sheath 100 is illustrated herein with the use of the blood pump 150, various other medical devices may be used in conjunction with the introducer sheath 100.

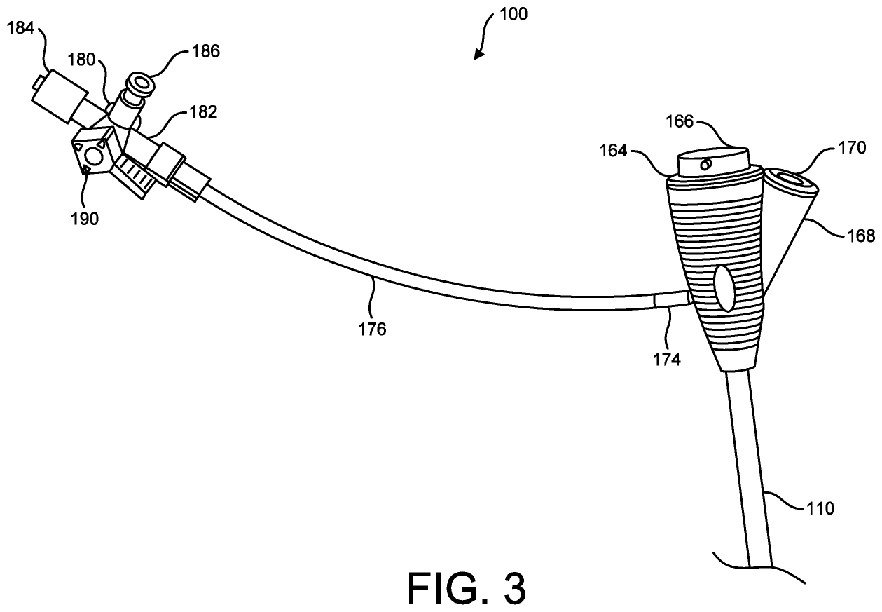

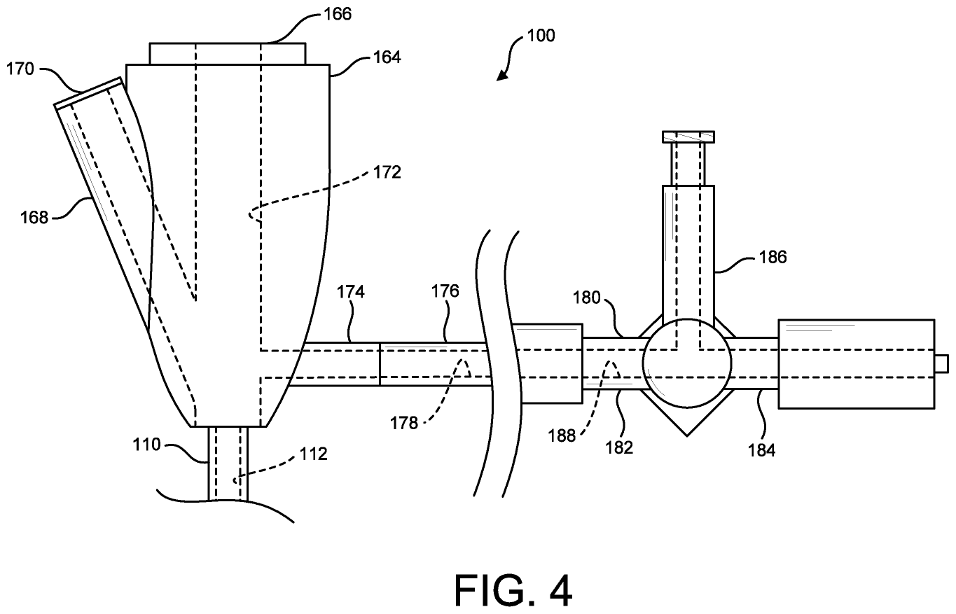

FIGS. 3 and 4 illustrate the introducer sheath 100 in further detail. As illustrated, the hub 120 includes a first arm 164 having a first port 166 and a second arm 168 having a second port 170. Both the first port 166 and the second port 170 are configured to couple to medical devices and permit the medical devices to extend into a hub lumen 172 (FIG. 4). The hub lumen 172 is coupled to the body portion lumen 112 (FIG. 4) and, as a result, the medical devices may be advanced through the body portion lumen 112 and toward the target location within the patient. The first port 166 and the second port 170 may also include internal seals to facilitate hemostasis.

With continued reference to FIGS. 3 and 4, the hub 120 further includes third port 174, which may also be referred to as an access port or a side arm. The third port 174 couples to an access conduit 176, which may also be referred to as a flush conduit or a flush line. The access conduit 176 includes an access lumen 178 (FIG. 4) coupled to the hub lumen 172. Opposite the third port 174, the access conduit 176 couples to a three-way stopcock 180. The stopcock 180 includes a first leg 182 that couples to the access conduit 176, a second leg 184, and a third leg 186. The first leg 182, the second leg 184, and the third leg 186 together define a stopcock lumen 188 (FIG. 4) coupled to the access lumen 178. The stopcock 180 also includes a lever 190 that is pivotable to selectively isolate one of the first leg 182, the second leg 184, and the third leg 186 from the others of the first leg 182, the second leg 184, and the third leg 186. These features of the stopcock 180 are described in further detail herein.

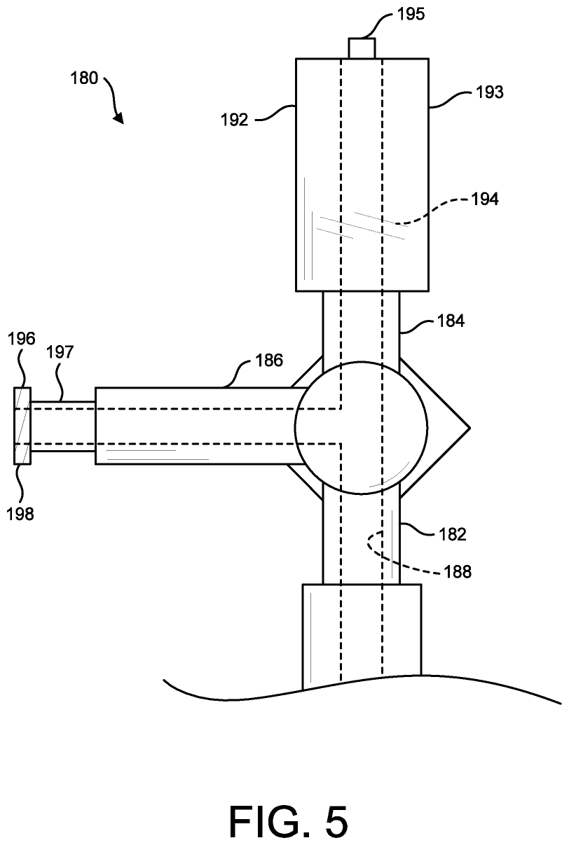

FIG. 5 illustrates the stopcock 180 in further detail. As illustrated, the second leg 184 of the stopcock 180 includes a male luer connector 192 (also referred to as a male luer lock connector and, generally, being a connector including an outer collar 193 with internal threads 194 and a spigot 195). The third leg 186 of the stopcock 180 includes a female luer connector 196 (also referred to as a female luer lock connector and, generally, being a connector including a spigot 197 with external threads 198).

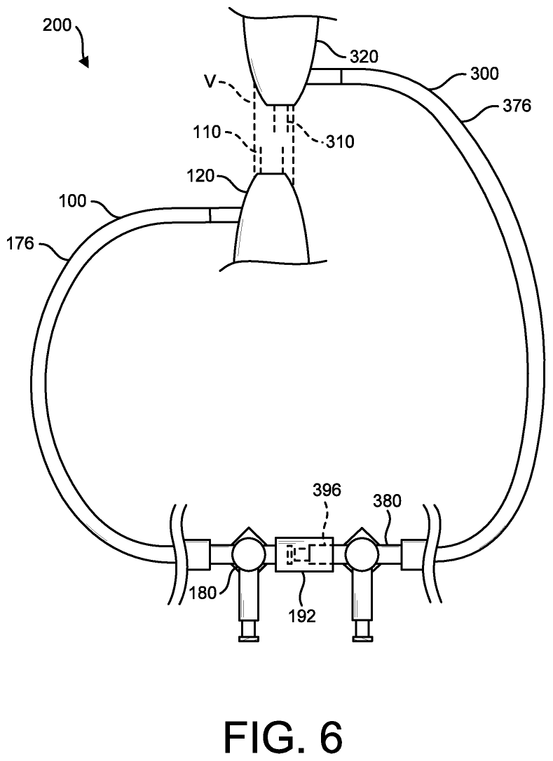

FIG. 6 illustrates a medical device assembly 200 that forms a bypass circuit for blood vessel V of a patient, such as the femoral artery. The medical device assembly 200 includes a first introducer sheath, more specifically the introducer sheath 100 described herein. The introducer sheath 100 includes the components and features described herein, including a first hub 120, a first body portion 110 coupled to the first hub 120, a first access conduit 176 coupled to the first hub 120, and a first three-way stopcock 180 coupled to the first access conduit 176, the first three-way stopcock 180 including a male luer connector 192. The first body portion 110 of the sheath 100 is positioned in the blood vessel V in a retrograde manner (that is, such that the first body portion 110 faces the opposite direction as blood flow in the blood vessel V). The medical device assembly 200 further includes a second introducer sheath 300, which may have the same or similar features as the first introducer sheath 100. Generally, the second introducer sheath 300 includes a second hub 320, a second body portion 310 coupled to the second hub 320, a second access conduit 376 coupled to the second hub 320, and a second three-way stopcock 380 coupled to the second access conduit 376, the second three-way stopcock 380 including a female luer connector 396. The female luer connector 396 couples to the male luer connector 192. The second body portion 310 of the sheath 300 is positioned in the blood vessel V in an antegrade manner (that is, such that the second body portion 110 faces the same direction as blood flow in the blood vessel V). As a result, blood in the blood vessel V flows into the first body portion 110, through the first hub 120, the first access conduit 176, the first stopcock 180, the second stopcock 380, the second access conduit 376, the second hub 320, the second body portion 310, and back into the blood vessel V.

By coupling the sheaths 100 and 300 via a male luer connector and a female luer connector, use of a separate connector, such as a male-to-male luer connector, is obviated.

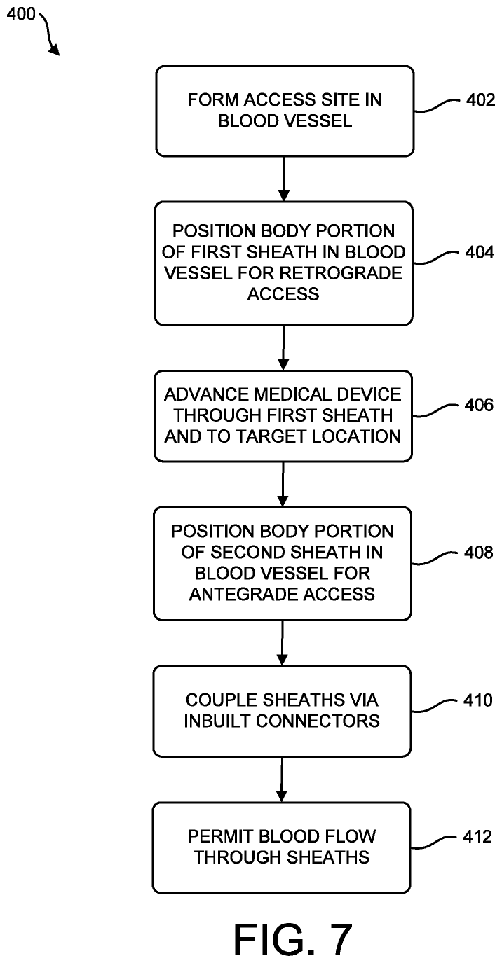

FIG. 7 illustrates a diagram of an exemplary method 400 of using a sheath assembly to deliver a medical device to a target location in a patient, in accordance with embodiments of the subject matter disclosed herein. The following description of the method 400 refers to the first sheath 100 and the second sheath 300 and their features as illustrated in FIG. 6, although it is understood that any sheaths contemplated herein may be used in a similar manner. At block 402, the method begins by forming an access site in a blood vessel of the patient. The blood vessel may be, for example, the femoral artery. At block 404, the first body portion 110 of the first sheath 100 is positioned in the blood vessel of the patient in a retrograde manner. At block 406, a medical device, such as the blood pump 150, is advanced through the first body portion 110 of the first sheath 100 and to a target location, more specifically a target cardiac location such as the left ventricle. The medical device may then be activated, more specifically the blood pump 150 may be energized to provide cardiac support to the patient. At block 408, the second body portion 310 of the second sheath 300 is positioned in the blood vessel of the patient in an antegrade manner. At block 410, the first sheath 100 and the second sheath 300 are coupled via the first inbuilt male luer connector 192 of the first sheath 100 and the female luer connector 396 of the second sheath 300. At block 412, blood flows through the bypass circuit formed by the first sheath 100 and the second sheath 300. More specifically, blood in the blood vessel flows into the first body portion 110, through the first hub 120, the first access conduit 176, the first stopcock 180, the second stopcock 380, the second access conduit 376, the second hub 120, the second body portion 310, and back into the blood vessel and provides adequate blood perfusion to the lower leg of the patient.

In some embodiments, the first sheath 100 and the second sheath 300 may have different sizes because the first sheath 100 receives the medical device and the second sheath 300 does not. That is, the first body portion 110 of the first sheath 100 may have a first size, the second body portion 310 of the second sheath 300 may have a second size, and the second size is less than the first size. For example, the first size may be 16 French and the second size may be 6 French. In other embodiments, the first sheath 100 and/or the second sheath 300 may have other sizes.

Various modifications and additions can be made to the exemplary embodiments discussed without departing from the scope of the present invention. For example, while the embodiments described herein refer to particular features, the scope of this invention also includes embodiments having different combinations of features and embodiments that do not include all of the herein-described features.

What is claimed is:

1. A sheath for receiving a medical device, the sheath comprising:
a hub comprising a hub lumen and a port coupled to the hub lumen, the port and the hub lumen arranged to receive the medical device;
a body portion coupled to the hub and configured to be disposed in a patient; the body portion comprising a body portion lumen coupled to the hub lumen, and the body portion lumen arranged to receive the medical device;
an access conduit coupled to the hub, the access conduit comprising an access lumen coupled to the hub lumen;
a stopcock coupled to the access conduit opposite the hub, the stopcock comprising:
a stopcock lumen coupled to the access lumen;
a first leg coupled to the access conduit;
a second leg comprising a male luer connector; and
a third leg comprising a female luer connector.

2. The sheath of claim 1, wherein the port is a first port, and the hub further comprises a second port.

3. The sheath of claim 1, wherein the stopcock lumen is defined within the first leg, the second leg, and the third leg, and the stopcock further comprises a lever being pivotable to selectively isolate one of the first leg, the second leg, and the third leg from the others of the first leg, the second leg, and the third leg.

4. The sheath of claim 1, wherein the body portion has a size of 16 French.

5. The sheath of claim 1, wherein the body portion has a size of 6 French.

6. A medical device assembly, comprising:
a first sheath comprising:
a first hub;
a first body portion coupled to the first hub, the first body portion arranged to be disposed in a patient;
a first access conduit coupled to the first hub;
a first three-way stopcock coupled to the first access conduit, the first three-way stopcock comprising a male luer connector;
a second sheath comprising:
a second hub;
a second body portion coupled to the second hub, the second body portion arranged to be disposed in the patient;
a second access conduit coupled to the second hub; and
a second three-way stopcock coupled to the second access conduit, the second three-way stopcock comprising a female luer connector arranged to be coupled to the male luer connector of the first sheath.

7. The medical device assembly of claim 6, wherein the first body portion has a first size, the second body portion has a second size, and the second size is less than the first size.

8. The medical device assembly of claim 7, wherein the first size is 16 French and the second size is 6 French.

9. The medical device assembly of claim 6, further comprising a medical device configured to be advanced through the first body portion.

10. The medical device assembly of claim 6, wherein the female luer connector is a first female luer connector, and the second three-way stopcock further comprises a second female luer connector.

11. A method of using a sheath assembly, the sheath assembly comprising a first sheath and a second sheath, the first sheath comprising a first body portion and a first stopcock, the first stopcock comprising a male luer connector, and the second sheath comprising a second body portion and a second stopcock, the second stopcock comprising a female luer connector, the method comprising:
positioning the first body portion of the first sheath in a blood vessel of a patient;
positioning the second body portion of the second sheath in the blood vessel of the patient;
coupling the first sheath and the second sheath via the male luer connector of the first sheath and the female luer connector of the second sheath;
receiving blood from the blood vessel in the first body portion of the first sheath;
providing blood from the first sheath to the second sheath via the first stopcock and the second stopcock; and
providing blood from the second body portion of the second sheath to the blood vessel.

12. The method of claim 11, further comprising advancing a medical device through the first body portion of the first sheath.

13. The method of claim 12, further comprising advancing the medical device to a target cardiac location.

14. The method of claim 13, wherein the target cardiac location is the left ventricle.

15. The method of claim 12, wherein the medical device is a blood pump.

16. The method of claim 11, wherein positioning the first body portion of the first sheath in the blood vessel of the patient comprises positioning the first body portion in a retrograde manner.

17. The method of claim 11, wherein positioning the second body portion of the second sheath in the blood vessel of the patient comprises positioning the second body portion in an antegrade manner.

18. The method of claim 11, wherein the first body portion has a first size, the second body portion has a second size, and the second size is less than the first size.

19. The method of claim 18, wherein the first size is 16 French and the second size is 6 French.

20. The method of claim 11, wherein the blood vessel is the femoral artery.
